(19) United States
(12) Patent Application Publication
(10) Pub. No.: US 2022/0056376 A1
(43) Pub. Date: Feb. 24, 2022

(54) REMOVAL OF ELECTROLUMINESCENCT MATERIALS FOR SUBSTRATES

(71) Applicant: 3M INNOVATIVE PROPERTIES COMPANY, St. Paul, MN (US)

(72) Inventors: Yumi Kwon, Gyeonggi-do (KR); David J. Lundberg, Cottage Grove, MN (US); Karl J. Manske, St. Anthony, MN (US)

(21) Appl. No.: 17/416,993

(22) PCT Filed: Dec. 23, 2019
(86) PCT No.: PCT/IB2019/061301

§ 371 Date: Jun. 21, 2021

Related U.S. Application Data
(60) Provisional application No. 62/784,893, filed on Dec. 26, 2018.

(51) Int. Cl. C11D 3/24; C09K 11/06

(52) U.S. Cl. CPC C11D 3/24; C09K 2211/1011; C09K 11/06

(57) ABSTRACT

A process for removing an electroluminescent material from a substrate. The process includes providing a substrate having an electroluminescent material disposed on a surface thereof. The process further includes contacting the substrate with a composition. The composition includes a hydroflourocompound an organic solvent that forms an azeotrope with the hydroflourocompound when mixed with the hydroflourocompound.

### FIELD

The present disclosure relates to compositions and methods for removing electroluminescent materials from substrates.

### BACKGROUND

Various compositions for removing electroluminescent materials from metal substrates are described in, for example, U.S. Pat. 7,073,518.

### SUMMARY

In some embodiments, a composition is provided. The composition includes a hydroflourocompound and an organic solvent that forms an azeotrope with the hydroflourocompound when mixed with the hydroflourocompound. The composition further includes an electroluminescent material.

In some embodiments, a process for removing an electroluminescent material from a substrate is provided. The process includes providing a substrate having an electroluminescent material disposed on a surface thereof. The process further includes contacting the substrate with a composition. The composition includes a hydroflourocompound an organic solvent that forms an azeotrope with the hydroflourocompound when mixed with the hydroflourocompound.

The above summary of the present disclosure is not intended to describe each embodiment of the present disclosure. The details of one or more embodiments of the disclosure are also set forth in the description below. Other features, objects, and advantages of the disclosure will be apparent from the description and from the claims.

### BRIEF DESCRIPTION OF THE FIGURES

FIGS. 1A and 1B are X-ray Photoelectron Spectroscopy (XPS) spectra of the front and back of a metal mesh coated with electroluminescent material prior to cleaning.

FIGS. 2A and 2B are XPS spectra of two areas of a metal mesh coated with electroluminescent material then cleaned with a composition of the prior art.

FIG. 3 is an XPS spectrum of a metal mesh coated with electroluminescent material then cleaned and rinsed using a multistep prior art process.

FIG. 4 is an XPS spectrum of a metal mesh coated with electroluminescent material then cleaned using a single step process of the present invention.

### DETAILED DESCRIPTION

In the organic light emitting diode (OLED) manufacturing process, it is common for electroluminescent materials to be deposited onto (or otherwise present on) a metal mask. In order for these metal masks to be reused, the electroluminescent materials must be removed. Currently, solvents like N-Methyl-2-pyrrolidone (NMP), cyclohexanone, or isopropyl alcohol (IPA) are used to remove the electroluminescent materials from the metal masks. Following removal of the electroluminescent materials, the solvents must then be removed from the metal masks (e.g., rinsed off) using deionized water or fluorinated solvents because the drying time for such solvents is unacceptably long due to their high boiling points. Moreover, use of such solvents is undesirable, generally, due to their unfavorable toxicity profiles and flash points.

Consequently, compositions and methods for removing electroluminescent materials from metal masks that (i) can be carried out in a single step process (that is, a process that may not require an additional rinsing step); and/or (ii) employ materials with favorable toxicity profiles and/or flash points, may be desirable.

As used herein, “fluoro-” (for example, in reference to a group or moiety, such as in the case of “fluoroalkylene” or “fluoroalkyl” or “fluorocarbon”) or “fluorinated” means partially fluorinated such that there is at least one carbon-bonded hydrogen atom

As used herein, “perfluoro-” (for example, in reference to a group or moiety, such as in the case of “perfluoroalkylene” or “perfluoroalkyl” or “perfluorocarbon”) or “perfluorinated” means completely fluorinated such that, except as may be otherwise indicated, there are no carbon-bonded hydrogen atoms replaceable with fluorine.

As used herein, the singular forms “a”, “an”, and “the” include plural referents unless the content clearly dictates otherwise. As used in this specification and the appended embodiments, the term “or” is generally employed in its sense including “and/or” unless the content clearly dictates otherwise.

As used herein, the recitation of numerical ranges by endpoints includes all numbers subsumed within that range (e.g. 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

In some embodiments, the present disclosure is directed to compositions for removing electroluminescent materials from a substrate (e.g., a metal mask of the type commonly used in the OLED manufacturing process). The composition may include one or more fluorocompounds and one or more organic solvents.

In some embodiments, suitable fluorocompounds may include hydrofluorocompounds (i.e., a plurality of carbon atoms, at least one fluorine atom, and at least one hydrogen atom). In some embodiments, suitable hydrofluorocompounds may include hydrofluoroether compounds. In some embodiments, suitable hydrofluorocompounds or hydrofluoroether compounds may have a surface tension (at room temperature) of between 10 and 20 mN/m, between 12 and 17 mN/m, or between 13 and 16 mN/m. In some embodiments, suitable hydrofluorocompounds or hydrofluoroethers may have a boiling point of less than 100 degrees Celcius or less than 70 degrees Celcius.

In some embodiments, suitable hydrofluoroethers may include C₂F₅CF(OCH₃)CF(CF₃)₂, (CF₃)₂CFCF₂(OCH₂CH₃), (CF₃)₂CFCF₂OCH₃, or combinations thereof.

In some embodiments, suitable organic solvents may include any organic fluid that will form an azeotrope with the hydroflourocompounds of the composition. In some embodiments, suitable organic solvents may include 1-bromopropane, hexamethyldisilazane, isobutyl acetate, methylisobutyl ketone, trans-1,2-dichloroethylene, trifluoromethylbenzene, methanol, ethanol, isopropanol, t-butanol, hexafluoro-2-propanol, trifluoroethanol, pentafluoropropanol, 1-chlorobutane, 1,2-dihloropropane, 2,2-dichloropropane, 2-chlorobutane, i-butyl chloride, t-butyl chloride, heptane, iso-octane, cyclohexane, methyl cyclohexane, t-amylmethly ether, 1,2 dimethoxyethane, tetrahydrofuran, methyl ethyl ketone, acetonitrile, hexamethyl disoloxane, ethyl acetate, or combinations thereof. In some embodiments, the organic solvent may include or consist essentially of trans-1,2-dichloroethylene.

In some embodiments, the compositions may include any of the above-described hydrofluorocompounds in an amount of at least 5 wt. %, at least 10 wt. %, or at least 20 wt. %; or between 5 and 60 wt. %, between 10 and 50 wt. %, or between 20 and 40 wt. %, based on the total amount of hydrofluorocompounds and organic solvent in the composition. In some embodiments, the compositions may include organic solvents in an amount of at least 40 wt. %, at least 50 wt. %, or at least 60 wt. %; or between 40 and 95 wt. %, between 50 and 90 wt. %, or between 50 and 80 wt. %, based on the total amount of hydrofluorocompound and organic solvent in the composition.

In some embodiments. the compositions of the present disclosure may include a blend of C₂F₅CF(OCH₃)CF(CF₃)₂ and trans-1,2-dichloroethylene, such as such blends available under the trade designation NOVEC 73DE, 72DE, 72DA, or 71DE, all available from 3M Company of St. Paul, Minn.

In some embodiments, the above-described compositions may form azeotrope compositions (i.e., a multi-component composition that behaves like a single component in that the vapor produced by partial evaporation of the liquid at its boiling point has the same (or substantially same) composition as the liquid). In some embodiments, the azeotrope compositions may include any of those described in U.S. Pat. No. 7,071,154, which is herein incorporated by reference in its entirety.

It is to be appreciated that the compositions of the present disclosure have been discovered to adequately remove electroluminescent materials (e.g., OLED dyes) from the surface of metal substrates and are associated with significantly shorter drying times relative to the materials conventionally employed in metal mask cleaning processes (e.g, NMP, cyclohexanone, and IPA).

In some embodiments, the present disclosure is further directed to the above-described compositions, in their post-clean state. In this regard, the present disclosure is directed to any of the above-described cleaning compositions that include one or more electroluminescent materials dissolved, dispersed, or otherwise contained therein. In some embodiments, the electroluminescent materials may include any highly conjugated dye that responds to electric stimulation (such as those often employed in the OLED manufacturing process). In some embodiments, the electroluminescent materials may include copper (II) phthalocyanine, iridium, or platinum. In some embodiments, the electroluminescent materials may be present in the post-clean compositions in an amount of at least 0.001 wt. % or at least 0.01 wt. %., based on the total weight of fluoroethers and organic solvents present in the composition.

In some embodiments, the fluorine content in the compositions of the present disclosure may be sufficient to make the compounds non-flammable according to ASTM D-3278-96 e-1 test method (“Flash Point of Liquids by Small Scale Closed Cup Apparatus”).

In some embodiments, the compositions of the present disclosure have favorable toxicity profiles. More specifically, the compositions of the present disclosure may toxicity profiles that are more favorable than those of materials commonly employed to clean electroluminescent materials from metal masks (e.g., NMP).

In some embodiments, the present disclosure is further directed to working fluids that include the above-described compositions as a major component. For example, the working fluids may include at least 25%, at least 50%, at least 70%, at least 80%, at least 90%, at least 95%, or at least 99% by weight of the above-described compositions, based on the total weight of the working fluid.

In some embodiments, the present disclosure is further directed to methods of cleaning metal substrates. More specifically, in some embodiments, the present disclosure is further directed to methods of removing electroluminescent materials from metal masks (such as those commonly used in the OLED manufacturing process). In some embodiments, the method first includes providing a metallic substrate (e.g., a metal mask) having an electroluminescent material disposed on an external surface of the substrate. The electroluminescent material may be disposed on the external surface in a layer having a thickness of at least 10,000 Angstroms, at least 15,000 Angstroms, or at least 20,000 Angstroms.

In some embodiments, the method may then include providing any of the above described compositions. The method may then include contacting the electroluminescent material containing metal mask with the composition. In some embodiments, the compositions of the present disclosure can be used in either the gaseous or the liquid state (or both), and any of known or future techniques for “contacting” the substrate can be utilized. For example, a liquid cleaning composition can be sprayed or brushed onto the substrate, a gaseous cleaning composition can be blown across the substrate, or the substrate can be immersed (partially or completely) in either a gaseous or a liquid composition. Elevated temperatures, ultrasonic energy, and/or agitation can be used to facilitate the cleaning. In some embodiments, the methods of the present disclosure are carried out at room temperature. Various different cleaning techniques are described by B. N. Ellis in Cleaning and Contamination of Electronics Components and Assemblies, Electrochemical Publications Limited, Ayr, Scotland, pages 182-94 (1986), which is herein incorporated by reference in its entirety.

### Listing of Embodiments

1. A composition comprising:

a hydroflourocompound;

an organic solvent that forms an azeotrope with the hydroflourocompound when mixed with the hydroflourocompound; and

and an electroluminescent material.
2. The composition of embodiment 1, wherein the hydroflourocompound has a surface tension of between 12 and 17 mN/m.
3. The composition of any one of the previous embodiments, hydroflourocompound is a hydrofluoroether.
4. The composition of any one of the previous embodiments, wherein the hydrofluoroether is C₂F₅CF(OCH₃)CF(CF₃)₂.
5. The composition of any one of the previous embodiments, wherein the hydroflourocompound is present in the composition in an amount of between 5 and 60 wt. %, based on the total weight of hydrofluoroether and organic solvent present in the composition.
6. The composition of any one of the previous embodiments, wherein the organic solvent comprises 1-bromopropane, hexamethyldisilazane, isobutyl acetate, methylisobutyl ketone, trans-1,2-dichloroethylene, trifluoromethylbenzene, methanol, ethanol, isopropanol, t-butanol, hexafluoro-2-propanol, trifluoroethanol, pentafluoropropanol, 1-chlorobutane, 1,2-dihloropropane, 2,2-dichloropropane, 2-chlorobutane, i-butyl chloride, t-butyl chloride, heptane, iso-octane, cyclohexane, methyl cyclohexane, t-amylmethly ether, 1,2 dimethoxyethane, tetrahydrofuran, methyl ethyl ketone, acetonitrile, hexamethyl disoloxane, ethyl acetate, or combinations thereof.
7. The composition of any one of the previous embodiments, wherein the organic solvent comprises trans-1,2-dichloroethylene.
8. The composition of any one of the previous embodiments, wherein the organic solvent is present in the composition in an amount of between 40 and 95 wt. %, based on the total weight of fluoroether and organic solvent present in the composition.
9. The composition of any one of the previous embodiments, wherein trans-1,2-dichloroethylene is present in the composition in an amount of between 40 and 95 wt. %, based on the total weight of fluoroether and organic solvent present in the composition.
10. The composition of any one of the previous embodiments, wherein the composition is non-flammable according to ASTM D-3278-96 e-1 test method.
11. The composition of any one of the previous embodiments, wherein the electroluminescent material comprises copper (II) phthalocyanine, iridium, or platinum.
12. A process for removing an electroluminescent material from a substrate, the process comprising the steps of:

providing a substrate having an electroluminescent material disposed on a surface thereof;

contacting the substrate with a composition comprising:
a hydroflourocompound; and
an organic solvent that forms an azeotrope with the hydroflourocompound when mixed with the hydroflourocompound.
13. The process of embodiment 12, wherein the hydroflourocompound has a surface tension of between 12 and 17 mN/m.
14. The process of any one of embodiments 13-14, wherein the hydroflourocompound is a hydrofluoroether.
15. The process of embodiment 15, wherein the hydrofluoroether is C₂F₅CF(OCH₃)CF(CF₃)₂.
16. The process of any one of embodiments 12-15, wherein the hydroflourocompound is present in the composition in an amount of between 5 and 60 wt. %, based on the total weight of hydroflourocompound and organic solvent present in the composition.
17. The process of any one of embodiments 12-16, wherein the organic solvent comprises 1-bromopropane, hexamethyldisilazane, isobutyl acetate, methylisobutyl ketone, trans-1,2-dichloroethylene, trifluoromethylbenzene, methanol, ethanol, isopropanol, t-butanol, hexafluoro-2-propanol, trifluoroethanol, pentafluoropropanol, 1-chlorobutane, 1,2-dihloropropane, 2,2-dichloropropane, 2-chlorobutane, i-butyl chloride, t-butyl chloride, heptane, iso-octane, cyclohexane, methyl cyclohexane, t-amylmethly ether, 1,2 dimethoxyethane, tetrahydrofuran, methyl ethyl ketone, acetonitrile, hexamethyl disoloxane, ethyl acetate, or combinations thereof.
18. The process of any one of embodiments 12-17, wherein the organic solvent comprises trans-1,2-dichloroethylene.
19. The process of any one of embodiments 12-18, wherein the organic solvent is present in the composition in an amount of between 40 and 95 wt. %, based on the total weight of hydroflourocompound and organic solvent present in the composition.
20. The process of any one of embodiments 12-19, wherein trans-1,2-dichloroethylene is present in the composition in an amount of between 40 and 95 wt. %, based on the total weight of hydroflourocompound and organic solvent present in the composition.
21. The process of any one of embodiments 12-20, wherein the composition is non-flammable according to ASTM D-3278-96 e-1 test method.
22. The process of any one of embodiments 12-21, wherein the electroluminescent material comprises copper (II) phthalocyanine, iridium, or platinum.

### EXAMPLES

Objects and advantages of this disclosure are further illustrated by the following comparative and illustrative examples. Unless otherwise noted, all parts, percentages, ratios, etc. in the examples and the rest of the specification are by weight, and all reagents used in the examples were obtained, or are available, from general chemical suppliers such as, for example, Sigma-Aldrich Corp., Saint Louis, Mo., US or may be synthesized by conventional methods. The following abbreviations are used herein: min=minutes, nm=nanometers, wt %=percentage by weight.

| Material | Description | Source |
|---|---|---|
| Cyclohexanone | Hydrocarbon Cleaning Solvent | Sigma-Aldrich Corp, |
| | | St. Louis, MO, US |
| NOVEC | C₂F₅CF(OCH₃)CF(CF₃)₂/trans-1,2- | 3M Company, |
| 73DE | dichloroethylene fluid blend | St. Paul, MN, US |
| NOVEC 7100 | C₄F₉OCH₃ (blend of isomers) | 3M Company, |
| | | St. Paul, MN, US |
| NOVEC 7300 | C₂F₅CF(OCH₃)CF(CF₃)₂ | 3M Company, |
| | | St. Paul, MN, US |

### Cleaning Performance

Organic electroluminescent material A (EL-A) was deposited on coupons of metal mask in a vacuum chamber at 10⁻⁷ torr for 3 hours. EL-A was an indene-based electroluminescent material with UV absorbance peaks at 340 nm, 357 nm, 375 nm, and 396 nm, as determined by UV-Visible spectroscopy using a CARY 8454 UV-Vis Spectrophotometer available from Agilent Technologies, Santa Clara, CA, US. Cyclohexanone (Comparative Example CE1) and NOVEC 73DE (Example 1) were used as cleaning solutions in these tests. Samples were dipped into the cleaning solution for 1 minute, then removed. The surface of sample was observed immediately after removal from cleaning solution and after 5 min.

Both CE1 and Example 1 removed all the organic electroluminescent material EL-A from the metal mask surface. However, the coupon cleaned with Example 1 dried as soon it was removed from the Example 1 solution, whereas the cleaning fluid CE1 remained on the sample surface even after 5 min of drying.

### Comparison of Cleaning Methods

Samples of metal masks contaminated with organic electroluminescent material EL-A were cleaned using the three processes outlined below.
Comparative Example CE2: no cleaning performed
Comparative Example CE3: dipped in cyclohexanone for 1 min
Comparative Example CE4: dipped in cyclohexanone for 1 min, then NOVEC 7100 for 1 min

### Example 2

### Dipped in NOVEC 73DE for 1 Min

After all three cleaning process procedures, the organic electroluminescent material was removed from the surface. The samples were dried for 1 day at ambient conditions prior to surface analysis by X-ray Photoelectron Spectroscopy (XPS) using a K-ALPHA System from Thermo Scientific, Waltham, Mass., US to detect any residual cleaning agent.

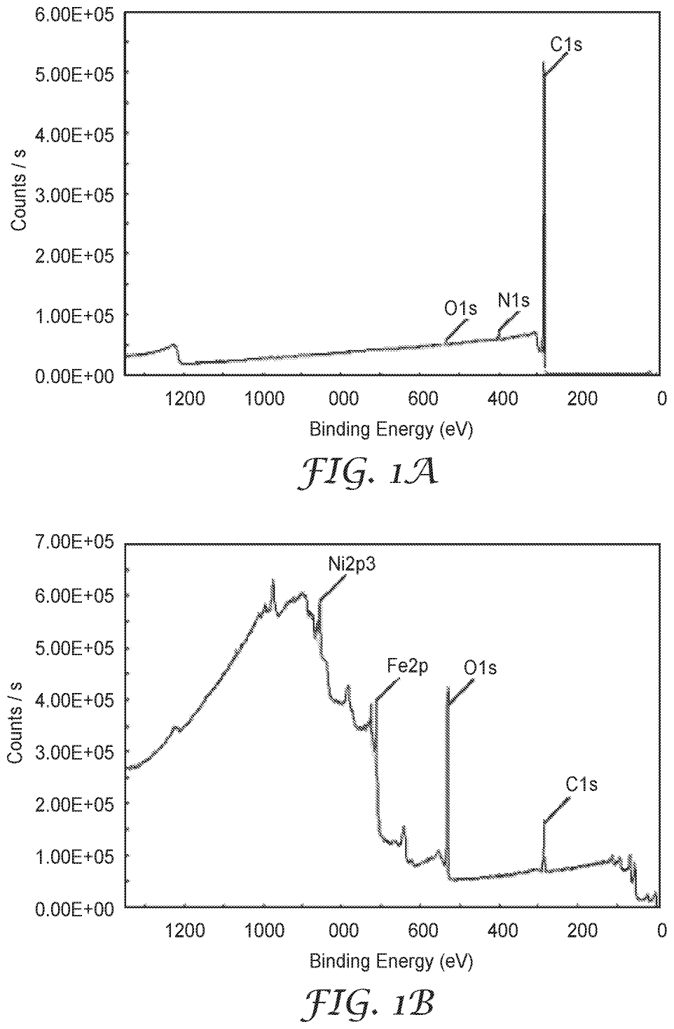

Surface analysis of both sides of uncleaned sample CE2 showed peaks corresponding to both organic electroluminescent material EL-A (deposition side, FIG. 1A) and the bare metal surface (non-deposition/bare metal side, FIG. 1B).

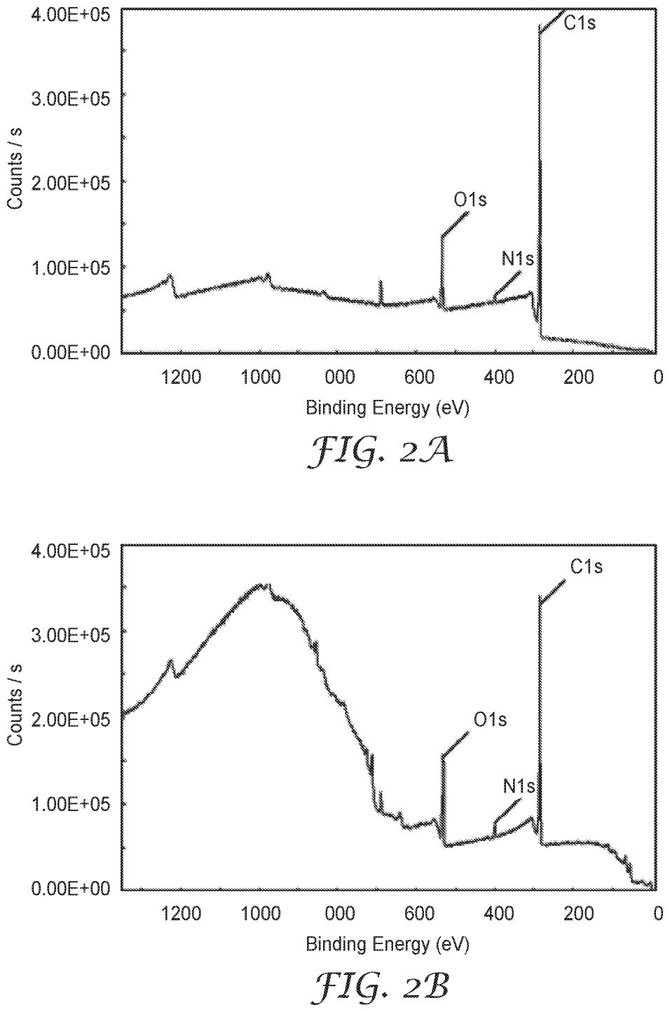

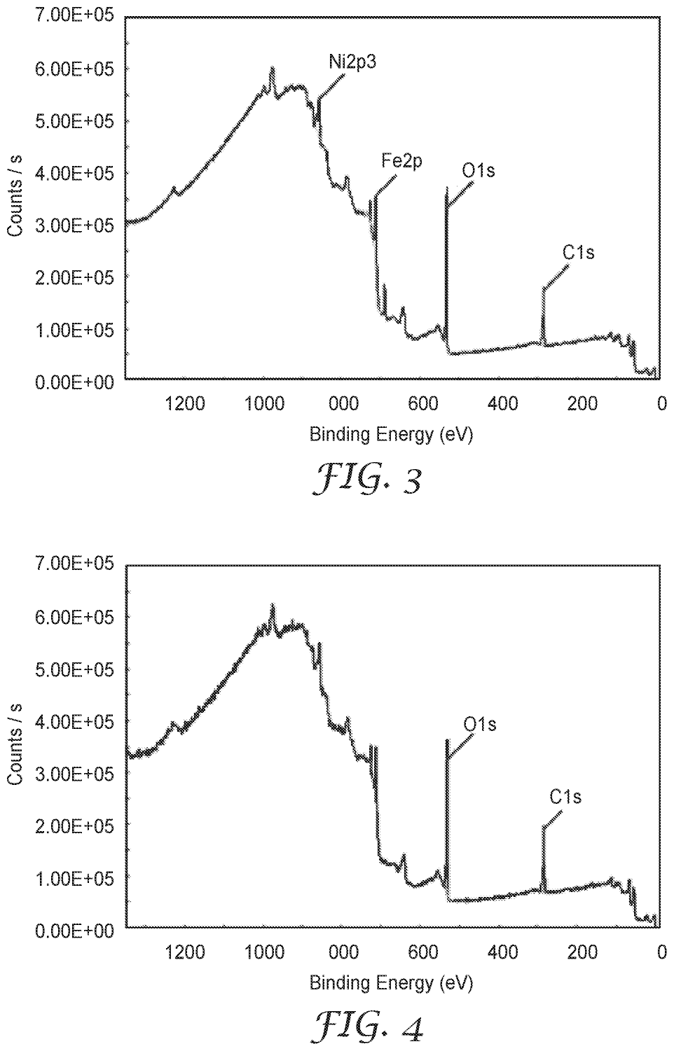

CE3 showed stains on the metal surface after cleaning, which were attributed to the slow drying rate of the cyclohexanone solvent. FIGS. 2A and 2B show XPS spectra of a stained region and unstained region, respectively. Both spectra show high intensity carbon peaks, indicating that the cyclohexanone solvent remained on the surface. XPS spectra of CE4 (FIG. 3) and Example 2 (FIG. 4) do not demonstrate high intensity of carbon peaks, and closely resemble the spectra for the bare metal surface of CE2 (FIG. 1B). Comparing FIGS. 2A, 2B, and 3, it appears that an additional rinsing using NOVEC 7100 removes residual cyclohexanone solvent remaining after the cleaning step. FIG. 4 (Example 2) further demonstrates that the present invention can achieve the same result in a single cleaning step.

### Effect of Cleaning Composition on Solubility of Organic Luminescent Material

Cleaning test solutions Examples 3-7 and Comparative Examples CE5 and CE6 were prepared by mixing NOVEC 7300 fluoroether and trans-1,2-dichloroethylene organic solvent in the amounts shown in Table 1.

TABLE 1
Composition of Cleaning Fluids

| | NOVEC 7300 | trans-1,2- |
|---|---|---|
| | (wt %) | dichloroethylene (wt %) |
| CE5 | 0 | 100 |
| Example 3 | 10 | 90 |
| Example 4 | 15 | 85 |
| Example 5 | 20 | 80 |
| Example 6 | 25 | 75 |
| Example 7 | 50 | 50 |
| CE6 | 100 | 0 |

0.5 wt % of organic electroluminescent material EL-A was added to each of the cleaning compositions in Table 1, the mixture was sonicated for 5 minutes, and the resulting material was observed with the unaided eye for haziness and undissolved particulate matter. CE5 and Examples 3-5 were clear liquids. Small particulates were observed on the top layer of Example 6. Example 7 was slightly opaque and CE6 was an opaque milky white.

1.0 wt % of organic electroluminescent material EL-B was added to each of the cleaning compositions in Table 1 and the resulting material was observed with the unaided eye for haziness and undissolved particulate matter. EL-B was an indene-based electroluminescent material with an UV absorbance peak at 345 nm, as determined by UV-Visible spectroscopy using a CARY 8454 UV-Vis Spectrophotometer available from Agilent Technologies, Santa Clara, Calif., US. EL-B appeared to dissolve relatively faster than EL-A in all of the cleaning solutions. EL-B dissolved immediately upon addition to CE5 and Examples 3-5, resulting in clear liquids. Example 7 was slightly hazy and CE6 was an opaque milky white.

Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows. All references cited in this disclosure are herein incorporated by reference in their entirety.

What is claimed is:

1. A composition comprising:
a hydroflourocompound;
an organic solvent that forms an azeotrope with the hydroflourocompound when mixed with the hydroflourocompound; and
and an electroluminescent material.

2. The composition of claim 1, wherein the hydroflourocompound has a surface tension of between 12 and 17 mN/m.

3. The composition of claim 1, hydroflourocompound is a hydrofluoroether.

4. The composition of claim 3, wherein the hydrofluoroether is C₂F₅CF(OCH₃)CF(CF₃)₂.

5. The composition of claim 2, wherein the hydroflourocompound is present in the composition in an amount of between 5 and 60 wt. %, based on the total weight of hydrofluoroether and organic solvent present in the composition.

6. The composition of claim 1, wherein the organic solvent comprises 1-bromopropane, hexamethyldisilazane, isobutyl acetate, methylisobutyl ketone, trans-1,2-dichloroethylene, trifluoromethylbenzene, methanol, ethanol, isopropanol, t-butanol, hexafluoro-2-propanol, trifluoroethanol, pentafluoropropanol, 1-chlorobutane, 1,2-dihloropropane, 2,2-dichloropropane, 2-chlorobutane, i-butyl chloride, t-butyl chloride, heptane, iso-octane, cyclohexane, methyl cyclohexane, t-amylmethly ether, 1,2 dimethoxyethane, tetrahydrofuran, methyl ethyl ketone, acetonitrile, hexamethyl disoloxane, ethyl acetate, or combinations thereof.

7. The composition of claim 1, wherein the organic solvent comprises trans-1,2-dichloroethylene.

8. The composition of claim 6, wherein the organic solvent is present in the composition in an amount of between 40 and 95 wt. %, based on the total weight of fluoroether and organic solvent present in the composition.

9. The composition of claim 7, wherein the trans-1,2-dichloroethylene is present in the composition in an amount of between 40 and 95 wt. %, based on the total weight of fluoroether and organic solvent present in the composition.

10. The composition of claim 1, wherein the composition is non-flammable according to ASTM D-3278-96 e-1 test method.

11. The composition of claim 1, wherein the electroluminescent material comprises copper (II) phthalocyanine, iridium, or platinum.

12. A process for removing an electroluminescent material from a substrate, the process comprising the steps of:
providing a substrate having an electroluminescent material disposed on a surface thereof;
contacting the substrate with a composition comprising:
a hydroflourocompound; and
an organic solvent that forms an azeotrope with the hydroflourocompound when mixed with the hydroflourocompound.

13. The process of claim 12, wherein the hydroflourocompound has a surface tension of between 12 and 17 mN/m.

14. The process of claim 12, wherein the hydroflourocompound is a hydrofluoroether.

15. The process of claim 14, wherein the hydrofluoroether is C₂F₅CF(OCH₃)CF(CF₃)₂.

16. The process of claim 12, wherein the hydroflourocompound is present in the composition in an amount of between 5 and 60 wt. %, based on the total weight of hydroflourocompound and organic solvent present in the composition.

17. The process of claim 12, wherein the organic solvent comprises 1-bromopropane, hexamethyldisilazane, isobutyl acetate, methylisobutyl ketone, trans-1,2-dichloroethylene, trifluoromethylbenzene, methanol, ethanol, isopropanol, t-butanol, hexafluoro-2-propanol, trifluoroethanol, pentafluoropropanol, 1-chlorobutane, 1,2-dihloropropane, 2,2-dichloropropane, 2-chlorobutane, i-butyl chloride, t-butyl chloride, heptane, iso-octane, cyclohexane, methyl cyclohexane, t-amylmethly ether, 1,2 dimethoxyethane, tetrahydrofuran, methyl ethyl ketone, acetonitrile, hexamethyl disoloxane, ethyl acetate, or combinations thereof.

18. The process of claim 12, wherein the organic solvent comprises trans-1,2-dichloroethylene.

19. The process of claim 17, wherein the organic solvent is present in the composition in an amount of between 40 and 95 wt. %, based on the total weight of hydroflourocompound and organic solvent present in the composition.

20. The process of claim 18, wherein the trans-1,2-dichloroethylene is present in the composition in an amount of between 40 and 95 wt. %, based on the total weight of hydroflourocompound and organic solvent present in the composition.

21. The process of claim 12, wherein the composition is non-flammable according to ASTM D-3278-96 e-1 test method.

22. The process of claim 12, wherein the electroluminescent material comprises copper (II) phthalocyanine, iridium, or platinum.
